(19) United States
(12) Patent Application Publication
(10) Pub. No.: US 2025/0123273 A1
(43) Pub. Date: Apr. 17, 2025

(54) SYSTEM TO CALCULATE HUMAN ORAL BIOAVAILABILITY

(71) Applicant: CN Bio Innovations Limited, Oxfordshire (GB)

(72) Inventors: Yassen Abbas, Cambridge (GB); Hailey Sze, Cambridge (GB); Tomasz Kostrzewski, Welwyn (GB); William Robert Thelin, Carrboro, NC (US); Elizabeth Mansfield Boazak, Durham, NC (US); Ashley Ann Spreen, Durham, NC (US)

(21) Appl. No.: 18/379,948

(22) Filed: Oct. 13, 2023

(51) Int. Cl. G01N 33/50; C12M 1/00; C12M 3/00; C12N 5/071

(52) U.S. Cl. CPC G01N 33/5082; C12M 21/08; C12M 23/34; C12M 29/10; C12N 5/0671; C12N 5/0679; C12N 5/0697; G01N 33/5038; C12N 2501/11; C12N 2513/00; C12N 2533/54

(57) ABSTRACT

The invention relates to methods and systems for the co-culture of primary liver cells and primary intestinal cells. In particular, the invention has application in estimating bioavailability.

### FIELD OF THE INVENTION

The present invention relates to methods and systems for co-culturing primary liver cells and primary intestinal cells. In particular, embodiments of the invention relate to the use of such systems to calculate bioavailability.

### BACKGROUND TO THE INVENTION

Cell culture refers to the growth of cells in an artificial controlled environment. Primary cell culture is the ex vivo culture of cells obtained directly from a multicellular organism as opposed to immortalised cell lines. Generally, primary cell culture is more representative of in vivo tissues than cell lines; however, it is often more difficult to achieve appropriate conditions to maintain primary cells in culture than cell lines.

Cell co-culture is used to study cross-talk between two or more populations of cells. Since in vivo there are usually multiple types of cells involved in any one process, it is important to use co-culture systems to gain an understanding of how different populations of cells interact and affect each other. Direct co-culture refers to culturing cells where said cells are in direct physical contact with each other. Indirect co-culture on the other hand refers to the culture of cells in different environments where different populations of cells are not in direct physical contact with each other.

Both the liver and intestines have critical roles in metabolism and the uptake of drugs, nutrients and metabolites that we consume orally. The liver comprises enzymes to metabolise such compounds while the intestines are the primary site of absorption for such compounds. Consequently, both liver and intestinal cells are key to determining the bioavailability of compounds such as drugs, metabolites and nutrients.

Bioavailability refers to the amount of a compound, such as a drug or metabolite which enters circulation. Oral bioavailability is defined as the amount of a compound that reaches systemic circulation after oral consumption, absorption through the intestinal tract and metabolism in the liver. The current industry standard to estimate oral bioavailability is through in vivo human clinical trials and animal studies, where a treatment is given orally and intravenously to allow oral bioavailability to be calculated. However, human clinical trials are incredibly expensive and it is therefore desirable to gauge an idea of predicted bioavailability before reaching this stage of research. This is usually done via animal studies; however, animal models are not always representative of humans when it comes to bioavailability, limiting the usefulness of such studies. In vitro studies are much cheaper and can be carried out with a much higher throughput than human and animal in vivo studies. In addition to this, the FDA will accept the use of in vitro methods to determine bioavailability of a compound. However, there are few methods suitable for such purposes. Although assays have been developed for the co-culture of liver and intestinal cells to estimate bioavailability, this has only been available for the co-culture of liver cell lines with intestinal cell lines as well as primary liver cells with intestinal cell lines. There has been a distinct absence of assays that can be used to co-culture primary liver cells with primary intestinal cells. This has been problematic as cell lines may not have the same metabolic activity or level of transporter expression as primary cells. This ultimately means that such models are unable to provide an accurate or reliable estimation of bioavailability. Therefore, there is a need for methods and systems for the co-culture of primary liver cells and primary intestinal cells that can be used to estimate bioavailability. The present invention addresses this need.

### SUMMARY OF THE INVENTION

The inventors have developed methods and systems suitable for the co-culture of primary liver cells and primary intestinal cells, with different media requirements to maintain functionality and metabolic activity of each cell type. In particular, these methods and systems have applications in performing studies to estimate bioavailability.

In one aspect of the present invention there is a method of co-culturing primary liver cells and primary intestinal cells, the method comprising;
a. Providing a system comprising at least one three compartment set comprising a liver compartment, an apical compartment and a basolateral compartment, wherein the system comprises a first fluid circulation path whereby the basolateral compartment is fluidically connected to the liver compartment, the first fluid circulation path being selectively interruptible so as to fluidically isolate the liver compartment from the basolateral compartment;
b. seeding primary liver cells in the liver compartment;
c. adding primary intestinal cells to the apical compartment wherein said primary intestinal cells form a barrier between the apical and basolateral compartments;
d. circulating a medium through the first fluid circulation path that does not comprise epidermal growth factor (EGF);
e. adding media to the apical compartment that comprises EGF.

The media in the apical compartment and the media in the liver and basolateral compartments should be considered separate i.e. fluid in the apical compartment does not pass into the liver compartment or the basolateral compartment. Only medium in the apical compartment comprises EGF. Medium in the liver compartment and the basolateral compartment does not comprise EGF.

In an embodiment, the first fluid circulation path comprises a mechanism such as but not limited to a switch, valve, pump or closure which can selectively interrupt the first fluid circulation path. When the mechanism in the first fluid circulation path is “active” or “switched on” the basolateral compartment is fluidically connected to the liver compartment. When the mechanism is “inactive” or “switched off” the basolateral compartment and the liver compartment are not fluidically connected.

In a preferred embodiment, the mechanism that can selectively interrupt the first fluid circulation path is an interconnecting pump.

In an embodiment, when the liver and basolateral compartments are fluidically connected, the system may further comprise a second and/or third fluid circulation path that recirculates media in the liver compartment and/or the basolateral compartment respectively.

In an embodiment, when the liver and basolateral compartments are fluidically isolated, the system may further comprise a second and/or third fluid circulation path that recirculates media in the liver compartment and/or the basolateral compartment respectively.

The second and/or third fluid circulation paths may comprise a mechanism such as but not limited to a switch, valve, pump or closure.

In an embodiment a liver pump recirculates media in the liver compartment and/or a basolateral pump recirculates media in the basolateral compartment.

In an embodiment, the three compartment set further comprises a second fluid circulation path wherein fluid in the liver compartment is recirculated when the liver pump is “active” or “switched on”. When the liver pump is “inactive” or “switched off” fluid in the second fluid circulation path is not recirculated. In an embodiment, recirculation of fluid in the liver compartment helps maintain hepatocyte functionality.

In an embodiment, the three compartment set further comprises a third fluid circulation path wherein fluid in the basolateral compartment is recirculated when the basolateral pump is “active” or “switched on”. When the basolateral compartment is “inactive or “switched off” fluid in the third fluid circulation path is not recirculated. In an embodiment, recirculation of fluid in the third circulation path aids mixing of components of said fluid.

In an embodiment, the three compartment set comprises a first fluid circulation path as described above.

In an embodiment, the three compartment set comprises a first and second fluid circulation path as described above.

In an embodiment, the three compartment set comprises a first and third fluid circulation path as described above.

In a preferred embodiment, the three compartment set comprises a first, second and third fluid circulation path as described above.

The media in the apical compartment and the media in the fluidically connected liver and basolateral compartments should be considered separate i.e. fluid in the apical compartment does not pass into the first, second or third fluid circulation paths and vice versa. Only medium in the apical compartment comprises EGF. Medium in the first, second and third fluid circulation paths does not comprise EGF.

In methods and systems of the present invention, the system comprises at least one three compartment set, at least two three compartment sets, at least three three compartment sets, at least four three compartment sets, at least five three compartment sets, at least six three compartment sets or more. Preferably, methods and systems of the present invention comprise at least six three compartment sets. Systems of the present invention may be used independently, or concurrently with additional systems.

In methods and systems of the present invention medium does not pass from the apical compartment to the liver compartment and/or the basolateral compartment.

In the context of the present invention medium added to the apical compartment may already comprise EGF. Alternatively, EGF may be added separately to the medium in the apical compartment. In other words, EGF may be added simultaneously with the medium or separately from the medium, however, both are considered to be adding medium comprising EGF.

In a preferred embodiment, the medium circulated through the first, second and third fluid circulation paths is the same as the medium in the apical compartment, except only the medium in the apical compartment comprises EGF.

In an alternative embodiment, the medium circulated through the first, second and third fluid circulation paths is different to the medium in the apical compartment and only the medium in the apical compartment comprises EGF.

In a preferred embodiment, the medium in the apical compartment comprises 0.1-1000 ng/ml EGF. More preferably, the medium in the apical compartment comprises 2.5-50 ng/ml EGF.

In some embodiments primary liver cells and/or said primary intestinal cells are seeded on a biomimetic scaffold or a 3D scaffold. Preferably, said primary liver cells are seeded on a porous collagen coated scaffold and/or primary intestinal cells are seeded on a biomimetic scaffold. Seeding primary liver cells and primary intestinal cells onto such a scaffold provides a culture environment which more closely resembles that of in vivo organs.

In an embodiment at least one of the apical compartment, the basolateral compartment and the liver compartment comprise a 3D scaffold. In the context of the present invention a 3D scaffold is a three-dimensional structure that provides a physical support for cells to grow. 3D scaffolds can be selected that vary in their porosity, stiffness and degradability. The skilled person would be able to select an appropriate 3D scaffold to meet their requirements. 3D scaffolds allow cells to grow in an environment which is more representative of what is seen in vivo. Biomimetic scaffolds refer to scaffolds comprising biomimetic materials. Biomimetic materials refer to materials that are designed to replicate the structure and characteristics of biological materials that occur in nature.

A variety of 3D scaffolds are available for use in cell culture, these include, but are not limited to: hydrogel scaffolds, nanofiber scaffolds, collagen scaffolds, polystyrene scaffolds and polycaprolactone scaffolds.

In an embodiment, the liver compartment comprises a porous collagen coated scaffold and/or the apical compartment comprises a biomimetic scaffold.

In an embodiment, primary liver cells and/or primary intestinal cells are seeded on a biomimetic scaffold or a 3D scaffold. Preferably, primary liver cells are seeded on a porous collagen coated scaffold and/or primary intestinal cells are seeded on a biomimetic scaffold.

In an embodiment, primary liver cells seeded in the liver compartment may comprise at least one cell type selected from hepatocytes, kupffer cells, sinusoidal endothelial cells, stellate cells or any other type of cell found in the liver. Preferably, said primary liver cells are human.

Primary intestinal cells seeded in the apical compartment and the basolateral compartment may comprise at least one cell type selected from enterocytes, goblet cells, paneth cells, enteroendocrine cells, or any other type of cell found in the intestines. Preferably, said primary intestinal cells are human.

In an embodiment primary intestinal cells are cultured in expansion medium and once said primary intestinal cells have formed a confluent monolayer they are then cultured in differentiation medium before being seeded in the apical compartment. Preferably, primary intestinal cells are cultured in expansion medium for approximately 8 days and subsequently cultured in differentiation medium for a further approximately 5 days. Expansion medium is a nutrient rich medium which promotes proliferation in order to obtain a large population of cells. Differentiation medium is a specialised medium containing specific molecules such as growth factors and hormones that induce cells to follow a specific lineage. Many types of expansion and differentiation media are commercially available. The skilled person would be able to choose appropriate expansion and differentiation media to suit their needs and may modify the culture time in expansion medium and differentiation medium to meet their specific requirements. In some embodiments, the method may not comprise this step at all.

In a preferred embodiment, primary liver cells are seeded into the liver compartment before primary intestinal cells are transferred to the apical compartment, preferably liver cells are seeded into the liver compartment approximately 4 days before primary intestinal cells are transferred to the apical compartment. More preferably, primary intestinal cells are seeded onto a biomimetic scaffold on a cell culture insert and are then cultured in expansion media for approximately 8 days before being transferred to differentiation media; and after approximately 5 days of culture in differentiation media the cell culture inserts are transferred into the apical compartment; wherein the transfer of the cell culture inserts with the primary intestinal cells occurs approximately 4 days after the liver cells are seeded into the liver compartment of the three compartment set.

In some embodiments the method further comprises adding at least one treatment to at least one of the apical compartment, the basolateral compartment and/or the liver compartment. Preferably, at least one treatment is added to the apical compartment and at least one treatment is added separately to the liver compartment. The skilled person would be able to select any treatment for their desired application. Examples of treatments that can be used with the present invention include but are not limited to: drugs, nutrients, metabolites, vitamins, cytokines, chemokines, hormones, lipids, carbohydrates, nucleic acids and peptides.

In an embodiment at least one sample of medium is taken from at least one of the apical compartment, the basolateral compartment and/or the liver compartment. Said sample or samples may be taken at the beginning, during, or at the end of the co-culture time course. One sample may be taken from one or more of the apical compartment, the basolateral compartment and/or the liver compartment. Preferably multiple samples are taken from one or more of the apical compartment, the basolateral compartment and/or the liver compartment, and more preferably multiple samples are taken from the apical and liver compartments.

In a preferred embodiment samples of media are taken from at least one of the apical compartment, the basolateral compartment and/or the liver compartment periodically. More preferably, samples of media are taken from the apical compartment and the liver compartment periodically. The skilled person would be able to select an appropriate time course for the co-culture and an appropriate frequency to periodically take samples to meet their specific needs.

In an embodiment, at least one treatment is added to at least one compartment following seeding of primary liver cells in the liver compartment and/or primary intestinal cells in the apical compartment. Preferably, at least one treatment is added to the liver compartment and in a separate three compartment set at least one treatment is added to the apical compartment following seeding of primary liver cells in the liver compartment and primary intestinal cells in the apical compartment. For primary co-culture, once both liver and intestinal cells have been added to at least one three compartment set of the system and preferably at least one treatment has been added to at least one compartment of the three compartment set of the system an experiment may be run for 1 hour, 2 hours, 3 hours, 4 hours, 5 hours, 6 hours, 7 hours, 8, hours, 9 hours, 10 hours, 11 hours, 12 hours, 24 hours, 36 hours, 48 hours, 60 hours, 72 hours, 84 hours, 96 hours, 108 hours, 120 hours, or more depending on the needs of the user. At least one sample of medium may be taken from at least one compartment every hour, every 2 hours, every 3 hours, every 4 hours, every 5 hours, every 6 hours, every 12 hours, every 24 hours, or at any other interval depending on the needs of the user.

In a preferred embodiment at least one treatment is added to the apical compartment and in a separate three compartment set, at least one treatment is added to the liver compartment following seeding of primary liver cells in the liver compartment and primary intestinal cells in the apical compartment and an experiment is run for 48 hours with at least one sample of media being taken at 0, 1, 4, 6, 24 and 48 hours from at least one of the liver compartment, the apical compartment and/or the basolateral compartment, more preferably said samples of media are taken from the apical compartment and the liver compartment.

In some embodiments, one sample is taken from the apical compartment of at least one three compartment set at the end of an experiment. In alternative embodiments, multiple samples are taken periodically from the apical compartment of at least one three compartment set.

Preferably, the at least one treatment added to the apical compartment is the same as the at least one treatment added to the liver compartment. More preferably, said treatment is added at a different concentration in the apical compartment compared to the concentration added to the liver compartment. Most preferably, said treatment is added to the apical compartment at a concentration higher than that of said treatment added to the liver compartment. Even more preferably, the treatment is added to the apical compartment at a concentration approximately 10 times higher than that of said treatment added to the liver compartment.

Preferably, samples of media taken from any of the apical compartment, the basolateral compartment and/or the liver compartment are analysed. Samples may be analysed for the levels of a treatment administered, metabolites produced, release of signalling molecules, levels of waste products, levels of cytokines or any other parameter of interest to the skilled person. Such parameters can be analysed using techniques known in the art, a non-limiting example of such a technique is LC-MS. LC-MS is an analytical chemistry technique that involves the separation of analytes followed by mass based detection. Analytes can be separated according to a variety of parameters. Partition chromatography separates analytes based on their solubility and hydrophobicity. Ion-exchange chromatography separates analytes based on their ionic charges. Size-exclusion chromatography exploits differences in the size of the analyte. Affinity chromatography separates analytes based on their affinity to bind with the stationary phase. The skilled person would be able to select an appropriate chromatography method to suit their needs and the particular analyte to be measured. This technique can be used to quantify the amount of a compound of interest and measure its concentration over time.

In an embodiment bioavailability of at least one treatment is calculated. Bioavailability is the proportion of a compound or substance that enters circulation after being introduced into the body.

Preferably, methods and systems of the present invention can be used to estimate oral bioavailability. Oral bioavailability is the proportion of an orally administered compound or substance that enters circulation.

In some embodiments the method comprises taking samples of media from at least one compartment periodically and measuring the concentration of said at least one treatment in said samples as described above. Preferably, the method further comprises calculating bioavailability of at least one treatment using the below formula:

$$Oral Bioavailability = \frac{area under the curve ( oral ) * dose ( IV )}{area under the curve ( IV ) * dose ( oral )}$$

Preferably, the dose is the concentration of at least one treatment in moles.

In an embodiment at least one treatment is added to the apical compartment of at least one three compartment set of the system and the apical compartment simulates an oral dosing regimen, and/or at least one treatment is added separately to the liver compartment of at least one three compartment set of the system and the liver compartment simulates an intravenous (IV) dosing regimen. Preferably, oral and IV dosing regimens are set up in separate three compartment sets concurrently. Oral and IV dosing regiments may be set up in separate three compartment sets within the same system, and/or separate three compartment sets within different systems.

In another aspect of the invention there is at least one system with at least two three compartment sets wherein the first three compartment set comprises;
a liver compartment comprising primary liver cells;
an apical compartment comprising primary intestinal cells; and
a basolateral compartment;

wherein said primary intestinal cells form a barrier between the apical and basolateral compartments and the three compartment set comprises a first fluid circulation path comprising a medium whereby the basolateral compartment is fluidically connected to the liver compartment, a second fluid circulation path comprising a medium whereby fluid in the liver compartment is recirculated, a third fluid circulation path whereby fluid in the basolateral compartment may be recirculated, and only the medium in the apical compartment comprises EGF;

and the second three compartment set comprises;
a liver compartment comprising primary liver cells;
an apical compartment;
a basolateral compartment;

wherein the apical and basolateral compartments do not comprise primary intestinal cells, and the three compartment set comprises a first fluid circulation path comprising a medium whereby the basolateral compartment is fluidically connected to the liver compartment, a second fluid circulation path comprising a medium whereby fluid in the liver compartment is recirculated, a third fluid circulation path whereby fluid in the basolateral compartment may be recirculated, and the medium in the liver and basolateral compartments does not comprise EGF.

In an embodiment, in an experiment simulating an oral dosing regimen, the first three compartment set is employed wherein a medium and primary liver cells are added to the liver compartment and a medium comprising EGF and primary intestinal cells are added to the apical compartment. Preferably, at least one treatment is added to the apical compartment of the three compartment set and the mechanism is active so that the liver compartment and the basolateral compartment are fluidically connected. In this embodiment, no treatment is added to the liver compartment.

In an embodiment, in an experiment simulating an intravenous dosing regimen, the second three compartment set is employed, wherein a medium and primary liver cells are added to the liver compartment. Preferably, at least one treatment is added to the liver compartment of the three compartment set and the mechanism is active so that the liver compartment and the basolateral compartment are fluidically connected. In this embodiment, primary intestinal cells are not seeded in the apical compartment, preferably the apical compartment does not comprise cells and no treatment is added to the apical compartment.

In an embodiment, experiments simulating an oral dosing regimen and experiments simulating an IV dosing regimen are carried out separately i.e. not in the same three compartment set at the same time. Preferably, multiple three compartment sets are used concurrently, some simulating an oral dosing regimen and some simulating an IV dosing regimen. In some embodiments multiple three compartment sets are used concurrently in the same system, some simulating an oral dosing regimen and some simulating an IV dosing regimen. In alternative embodiments, multiple three compartment sets are used concurrently across different systems, some simulating and oral dosing regimen and some simulating an IV dosing regimen.

In an embodiment, samples are taken from the liver compartment to calculate the area under the curve (AUC) for both of the oral and intravenous (IV) dosing regimens.

In an embodiment, samples are taken from the apical compartment to analyse absorption through the intestinal barrier and metabolism by primary intestinal cells.

The invention further provides a method of determining bioavailability, the method comprising;
a. providing at least one system comprising at least two three compartment sets comprising a liver compartment, an apical compartment and a basolateral compartment, wherein the three compartment set comprises a first fluid circulation path whereby the basolateral compartment is fluidically connected to the liver compartment, a second fluid circulation path whereby fluid in the liver compartment is recirculated, a third fluid circulation path whereby fluid in the basolateral compartment may be recirculated;
b. in the first three compartment set, seeding primary liver cells in the liver compartment and adding primary intestinal cells to the apical compartment wherein said primary intestinal cells form a barrier between the apical and basolateral compartments;
c. in the second three compartment set, seeding primary liver cells in the liver compartment and not adding primary intestinal cells to the apical compartment;
d. circulating medium that does not comprise EGF through the first, second and third fluid circulation paths of the first and second three compartment sets;
e. adding medium that does comprise EGF to the apical compartment of the first three compartment set;
f. adding at least one treatment to the apical compartment of the first three compartments set and separately adding at least one treatment to the liver compartment of the second three compartment set;
g. taking samples periodically from the apical compartment and the liver compartment of the first and second three compartment sets;
h. measuring the concentration of at least one treatment in said samples;
i. calculating bioavailability using the formula;

$$Oral Bioavailability = \frac{area under the curve ( oral ) * dose ( IV )}{area under the curve ( IV ) * dose ( oral )}$$

In an embodiment, samples are taken from the liver compartment in at least two three compartment sets to calculate the area under the curve for both of the oral and intravenous (IV) dosing regimens.

In an embodiment, samples are taken from the apical compartment to analyse absorption through the intestinal barrier and metabolism by primary intestinal cells.

Any number of the first and second three compartment sets may be used concurrently to meet the users experimental needs. In some embodiments, a plurality of three compartment sets are used concurrently in the same system. In alternative embodiments, a plurality of three compartment sets are used in multiple different systems. The user would be able to select the number of three compartment sets and the number systems to meet their specific needs.

In another aspect of the invention there is provided a system for the co-culture of primary liver cells and primary intestinal cells, wherein said system comprises at least one three compartment set comprising:
a liver compartment comprising primary liver cells;
an apical compartment optionally comprising primary intestinal cells; and
a basolateral compartment;

wherein said primary intestinal cells form a barrier between the apical and basolateral compartments and the three compartment set comprises a first fluid circulation path comprising a medium whereby the basolateral compartment is fluidically connected to the liver compartment, the first fluid circulation path being selectively interruptible so as to fluidically isolate the liver compartment from the basolateral compartment; the three compartment set may further comprise a second fluid circulation path comprising a medium whereby fluid in the liver compartment is recirculated, and/or a third fluid circulation path whereby fluid in the basolateral compartment may be recirculated, and only the medium in the apical compartment comprises EGF.

In an embodiment of the present invention medium in the first, second and third fluid circulation paths is not able to enter the apical compartment and medium in the apical compartment does not pass into the first, second, and third fluid circulation paths. Preferably, although media in first, second and third fluid circulation paths is not able to enter the apical compartment and vice versa, components of media from the first, second and third fluid circulation paths are able to be transported to the apical compartment and vice versa by active or passive transport.

In an embodiment, medium does not pass from the apical compartment of the system to the liver compartment.

In an embodiment, systems of the present invention are suitable for use in experiments simulating oral dosing regimens and/or experiments simulating IV dosing regimens as described above.

In a preferred embodiment a porous membrane separates the apical compartment and the basolateral compartment. More preferably said porous membrane separating the apical compartment and the basolateral compartment is in the form of a cell culture well insert. The skilled person would be aware of and be able to source suitable cell culture well inserts for their specific needs.

Porous membranes enable the partitioning of cellular microenvironments in vitro. Examples of porous membranes that are suitable for use with the present invention include but are not limited to polycarbonate (PC), polyethylene terephthalate (PET), and polytetrafluoroethylene (PTFE). Porous membranes are available in a variety of pore sizes, including but not limited to 0.4 μm, 1 μm, 3 μm and 8 μm. Preferably, a porous membrane is used with a pore size of 0.4 μm. Porous membranes are also available with a range of pore densities including but not limited to <0.85×10⁸, <1.70×10⁶ and <0.85×10⁵ (pores/cm²). The skilled person would be able to select an appropriate pore size and pore density for their specific needs. The material that can travel through the pores of the membrane is dependent on the pore size used.

In some embodiments at least one compartment comprises a 3D scaffold. Preferably, the liver compartment comprises a 3D scaffold. In some embodiments the liver compartment comprises a porous collagen coated scaffold and/or the apical compartment comprises a biomimetic scaffold.

Preferably, the apical compartment comprises primary jejunum stem/progenitor cells and the liver compartment comprises primary hepatocytes. More preferably the primary liver cells and primary intestinal cells are human.

A system according the present invention is a microphysiological system. A microphysiological system also known as organ-on-a-chip technology is a multi-channel 3D microfluidic cell culture, integrated circuit that simulates the activities, mechanics and physiological response of an entire organ or an organ system.

The apical compartment of the system according to the invention is representative of the internal lumen of the intestine. In an embodiment, this is established by seeding primary intestinal cells in the apical compartment, preferably onto a biomimetic scaffold on a porous membrane as described above.

In an embodiment primary intestinal cells in the apical compartment are exposed to differentiation cues. Such differentiation cues include but are not limited to growth factors, hormones and cytokines.

In an embodiment, primary intestinal cells form a polarised barrier between the apical compartment and the basolateral compartment.

The present invention is suitable to be scaled up to allow multiple experiments to be performed in parallel in a single system, or by the use of multiple systems concurrently.

### BRIEF DESCRIPTION OF THE FIGURES

FIG. 1—An example system for co-culturing primary liver cells and primary intestinal cells as disclosed herein, with an example experiment timeline.

FIG. 2—The metabolic rate of primary liver cell cultures in liver media only, serum free (SF) intestinal differentiation media (intestinal DM SF) and liver media at a ratio of 25:75, intestinal DM SF media and liver media at a ratio of 50:50 and intestinal DM SF media only after 4 days, and after 7 days.

FIG. 3-Intestinal barrier strength (measured by transepithelial electrical resistance) for primary intestinal cells cultured with the following media:
Media 1—WEM+cocktail B+500 nM hydrocortisone (HC) (negative control)
Media 2—Intestinal DM SF (positive control)
Media 3—WEM+cocktail B+500 nM hydrocortisone (HC)+50 ng/ml EGF+500 nM A83-01+1.25 mM N-acetylcysteine (NAC)
Media 4—Intestinal DM SF+1 uM hydrocortisone (HC)
Media 5—WEM+cocktail B+500 nM hydrocortisone (HC)+500 nM A83-01+1.25 mM N-acetylcysteine (NAC)
Media 6—Advanced DMEM/F12+cocktail B+500 nM hydrocortisone (HC)+50 ng/ml EGF+500 nM A83-01+1.25 mM N-acetylcysteine (NAC)
Media 7—Advanced DMEM+cocktail B+500 nM hydrocortisone (HC)+5 ng/ml EGF+500 nM A83-01+1.25 uM N-acetylcysteine (NAC)
Media 8—WEM+cocktail B+500 nM hydrocortisone (HC)+500 nM A83-01

FIG. 4—CYP3A4 metabolism in primary liver cells subjected to co-culture with primary intestinal cells with the following media:
Media 1-(Liver media): WEM+cocktail B+500 nM hydrocortisone (HC)
Media 2—Advanced DMEM+4% v/v cocktail B+500 nM hydrocortisone (HC)+2.5 ng/ml EGF+500 nM A83-01+1.25 uM N-acetylcysteine (NAC)
Media 3—Advanced DMEM+4% v/v cocktail B+500 nM hydrocortisone (HC)+5 ng/ml EGF+500 nM A83-01+1.25 uM N-acetylcysteine (NAC)
Media 4—Advanced DMEM+4% v/v cocktail B+500 nM hydrocortisone (HC)+10 ng/ml EGF+500 nM A83-01+1.25 uM N-acetylcysteine (NAC)
Media 5—
   System volume (liver and basolateral compartments)—Advanced DMEM+cocktail B+500 nM hydrocortisone (HC)+500 nM A83-01+1.25 uM N-acetylcysteine (NAC)
   Apical compartment-Advanced DMEM+cocktail B+500 nM hydrocortisone (HC)+50 ng/ml EGF+500 nM A83-01+1.25 uM N-acetylcysteine (NAC)

For media 2-4 the same media was used in the system volume (the liver and basolateral compartments) and the apical compartment.

FIG. 5—Intestinal barrier strength (measured by transepithelial electrical resistance) for primary intestinal cells cultured in the following media:
Media 1 (Liver media)—WEM+cocktail B+500 nM hydrocortisone (HC)
Media 2—Advanced DMEM+4% v/v cocktail B+500 nM hydrocortisone (HC)+2.5 ng/ml EGF+500 nM A83-01+1.25 uM N-acetylcysteine (NAC)
Media 3—Advanced DMEM+4% v/v cocktail B+500 nM hydrocortisone (HC)+5 ng/ml EGF+500 nM A83-01+1.25 uM N-acetylcysteine (NAC)
Media 4—Advanced DMEM+4% v/v cocktail B+500 nM hydrocortisone (HC)+10 ng/ml EGF+500 nM A83-01+1.25 uM N-acetylcysteine (NAC)
Media 5—
   System volume (liver and basolateral compartments)—Advanced DMEM+cocktail B+500 nM hydrocortisone (HC)+500 nM A83-01+1.25 uM N-acetylcysteine (NAC)
   Apical compartment-Advanced DMEM+cocktail B+500 nM hydrocortisone (HC)+50 ng/ml EGF+500 nM A83-01+1.25 uM N-acetylcysteine (NAC)

For media 2-4 the same media was used in the system volume (the liver and basolateral compartments) and the apical compartment.

FIG. 6-A schematic of the culture system and the fluid paths. The apical, basolateral and liver compartments are shown. There is independent fluid recirculation in the basolateral and liver compartments and the fluidic path between the basolateral and liver compartments can be connected via an interconnecting pump.

### LIST OF DEFINITIONS

The following definitions are used in the present description and claims to define the stated subject matter. Other terms not cited below are meant to have the generally accepted meaning in the field.

“Primary liver cells” are cells that have been directly isolated from liver tissue. The liver comprises multiple cell types, these include but are not limited to hepatocytes, kupffer cells, sinusoidal endothelial cells and stellate cells. Hepatocytes are the most abundant cell type in the liver and carry out a range of functions including but not limited to carbohydrate, lipid and protein metabolism, protein synthesis and detoxification of harmful substances. Kupffer cells are liver resident macrophages. Their main function is phagocytosis to remove foreign particles and microorganisms. Kupffer cells also produce inflammatory cytokines in response to infection. Sinusoidal endothelial cells line the liver sinusoids and help regulate the exchange of substances between the blood and the liver. Stellate cells are present in the perisinusoidal space (or space of Disse) (the space between the hepatocytes and sinusoidal epithelial cells) and are the liver's resident fibroblasts that store vitamin A and produce collagen. Stellate cells can become activated in response to injury, leading to the production of more collagen and fibrosis of the liver. In the context of the present invention primary liver cells can include one or more type of cell selected from hepatocytes, kupffer cells, sinusoidal endothelial cells, stellate cells or any other type of cell found in the liver.

“Primary intestinal cells” are cells that have been directly isolated from intestinal tissue. The intestines comprise multiple cell types, these include but are not limited to: enterocytes, goblet cells, paneth cells, enteroendocrine cells and stem cells. Enterocytes are the most abundant cell type in the small intestine and are responsible for absorbing compounds that have been consumed orally. Goblet cells secrete mucus, helping to protect the intestinal lining. Paneth cells secrete antibacterial substances, helping to protect from infection. Enteroendocrine cells secrete hormones that regulate digestion among other processes. Stem cells are located at the base of the intestinal crypts and are responsible for replenishing the other types of cell found in the intestines. In the context of the present invention primary intestinal cells can include one or more type of cell selected from enterocytes, goblet cells, paneth cells, enteroendocrine cells, or any other type of cell found in the intestines.

“Cell culture” refers to the cultivation of cells outside a living organism under controlled conditions, for example, temperature, pH, nutrient, and waste levels. Both eukaryotic and prokaryotic cells can be subjected to cell culture.

“Primary cell culture” refers to culturing cells that have been directly obtained from a multicellular organism.

“Co-culture” refers to a cell cultivation set-up in which at least two populations of cells are cultured with some degree of contact between them. Co-culture of cells is fundamental in studying cell-cell interactions. There are two forms of co-culture known in the art, namely direct and indirect co-culture.

“Direct co-culture” refers to culture of at least two populations of cells with direct physical contact between them.

“Indirect co-culture” refers to culture of at least two populations of cells together, but where different populations of cells are not in direct contact with each other. In an embodiment, the present invention is used for indirect co-culture of primary liver cells and primary intestinal cells.

“Fluidically connected” as used in the present description means that fluid is able to flow from one compartment to another. In the context of the present invention fluid is able to flow between the basolateral compartment and the liver compartment.

“Bioavailability” is the amount of a compound, such as a drug or metabolite which enters circulation.

“Oral bioavailability” is the amount of a compound that reaches systemic circulation after oral consumption. Oral bioavailability can be calculated using the following formula:

$$Oral Bioavailability = \frac{area under the curve ( oral ) * dose ( IV )}{area under the curve ( IV ) * dose ( oral )}$$

### DETAILED DESCRIPTION OF EXAMPLES

While the foregoing disclosure provides a general description of the subject matter encompassed within the scope of the present invention, including methods as well as the best mode of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is now described in the following non-limiting examples.

### Example 1—One Proposed Experimental Design

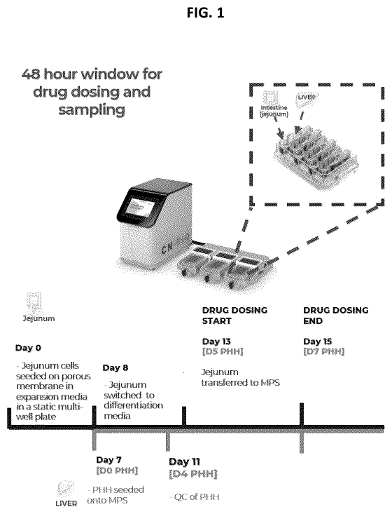

FIG. 1 shows a non-limiting example experimental design for use of the methods and systems of the present invention. According to this example primary intestinal cells would be first cultured on a conventional static cell culture plate in expansion media for 8 days before being transferred to differentiation media. On day 7 primary liver cells (PHH) are seeded into the liver compartment of the system of the invention. After 14 days the primary intestinal cells would be transferred to the apical compartment of the system of the invention. Once both the primary intestinal cells and the primary liver cells are in the apical and liver compartments respectively a treatment can be administered and samples taken from each compartment to be analysed over a time course, in this example 48 hours.

FIG. 6 shows a schematic of the system employed for such experiments. The apical, basolateral and liver compartments are shown. There is independent fluid recirculation in the basolateral and liver compartments and the fluidic path between the basolateral and liver compartments can be connected via an interconnecting pump. For experiments simulating an oral dosing regimen at least one treatment is added to the apical compartment. For experiments simulating an IV dosing regimen at least one treatment is added to the liver compartment in a separate system to that simulating an oral dosing regimen.

### Example 2—Intestinal Differentiation Media (Serum Free) is Detrimental to the Metabolic Functionality of Primary Liver Cells

Human primary liver cells were co-cultured with human primary intestinal cells. When this was done with liver cell media intestinal barrier function was compromised and when this was done with intestinal cell media the metabolic functionality of liver cells was eliminated.

To verify that it was the presence of intestinal cell media that was causing elimination in human primary liver cell functionality rather than the presence of human primary intestinal cells themselves, primary human hepatocytes (PHH) were cultured in isolation with either liver media, intestinal differentiation media or a mix of both of these media. The liver media used comprises William's E media (WEM), 4% cocktail B, for example, as available from ThermoFisher (comprising 0.5% penicillin-streptomycin, ITS+ (6.25 μg/ml insulin, 6.25 μg/ml transferrin, 6.25 ng/ml selenium complex, 1.25 mg/ml BSA, and 5.35 μg/ml linoleic acid), 2 mM GlutaMAX™, and 15 mMHEPES) and 500 nM hydrocortisone. The serum free intestinal differentiation media (intestinal DM SF) used comprised Advanced DMEM F12, 50 ng/ml EGF, 500 nM A83-01 (3-(6-Methyl-2-pyridinyl)-N-phenyl-4-(4-quinoliny)-1H-pyrazole-1-carbolhioamide) and 1.25 mM NAC.

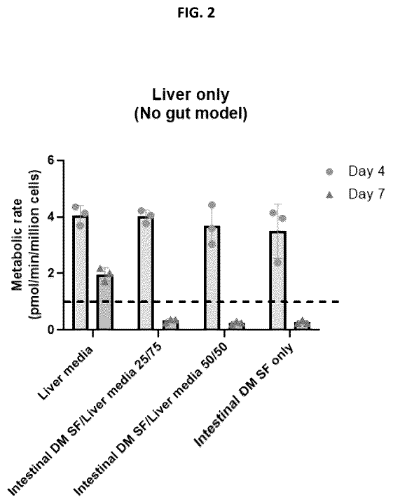

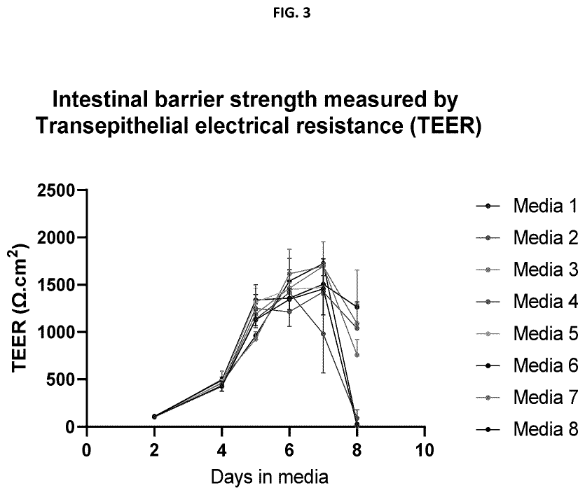

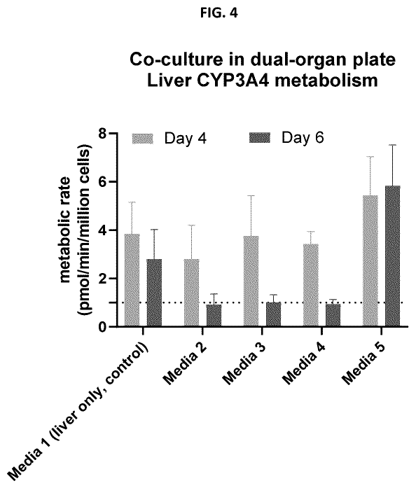

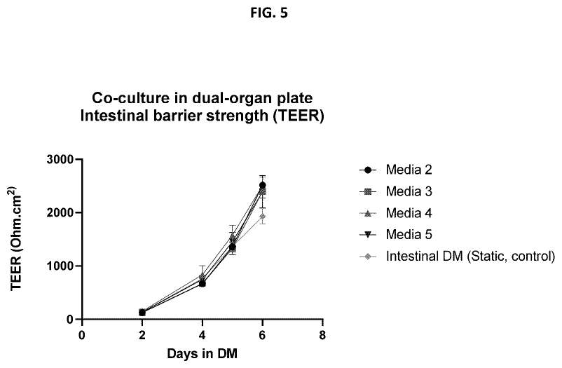

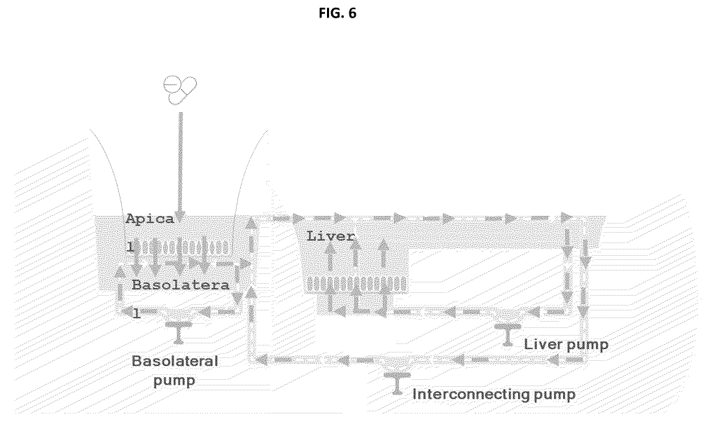

In order to measure the metabolic functionality of PHH the metabolic rate of CYP3A4 was measured. CYP3A4 is an important enzyme mainly found in the liver which metabolises a wide range of molecules such as drugs. The threshold for the metabolic rate of CYP3A4 was 1 pmol/min/million cells. If metabolic activity was lower than this, then this is insufficient and shows that the metabolic functionality of the PHH has been compromised. After 4 days in culture metabolic rate was high in all media compositions trialled, at approximately 4 pmol/min/million cells, although the lowest metabolic rate was seen in PHH cultured in only intestinal differentiation media. After 7 days in culture the metabolic rate of PHH cultured in liver media only was ˜2 pmol/min/million cells. However, in PHH cultured with intestinal differentiation media and liver media at a ratio of 25:75, intestinal differentiation media and liver media at a ratio of 50:50 and only intestinal differentiation media the metabolic rate had fallen to just ˜0.5 pmol/min/million cells after 7 days in culture, as illustrated in FIG. 2. This shows that intestinal differentiation media eliminates the metabolic functionality of PHH. Furthermore, it was surprisingly shown that the detrimental effects of intestinal differentiation media were not concentration dependent as metabolic rate was not increased in PHH that were cultured with a lower percentage of intestinal differentiation media.

### Example 3—EGF is Critical for Primary Intestinal Cell Barrier Function

It was clear that that serum free intestinal differentiation media compromised the metabolic functionality of primary liver cells, thus would not be appropriate for the co-culture of these two cell types. Therefore, the inventors investigated which components of serum free intestinal differentiation media were important for intestinal barrier function. To test this, primary human intestinal cells were cultured in media with different compositions as detailed below and transepithelial electrical resistance (TEER) was measured as an indicator of intestinal barrier strength.
Media 1—WEM+cocktail B+500 nM hydrocortisone (HC) (negative control)
Media 2—Intestinal DM SF (positive control)
Media 3—WEM+cocktail B+500 nM hydrocortisone (HC)+50 ng/mL EGF+500 nM A83-01+1.25 mM N-acetylcysteine (NAC)
Media 4—Intestinal DM SF+1 uM hydrocortisone (HC)
Media 5—WEM+cocktail B+500 nM hydrocortisone (HC)+500 nM A83-01+1.25 mM N-acetylcysteine (NAC)
Media 6—Advanced DMEM/F12+cocktail B+500 nM hydrocortisone (HC)+50 ng/mL EGF+500 nM A83-01+1.25 mM N-acetylcysteine (NAC)
Media 7—Advanced DMEM+cocktail B+500 nM hydrocortisone (HC)+5 ng/ml EGF+500 nM A83-01+1.25 uM N-acetylcysteine (NAC)
Media 8—WEM+cocktail B+500 nM hydrocortisone (HC)+500 nM A83-01

FIG. 3 shows that TEER was stable for Media 3, 4, 6 and 7 after 8 days in culture. Notably, all of these media comprise EGF, which had either been added independently or was included already as part of the intestinal DM SF media, the composition of which is given in Example 2. Meanwhile, TEER collapsed by 8 days in culture for Media 1, 2, 5 and 8. Media 1, 5 and 8 did not comprise EGF, whereas media 2 did comprise EGF but did not contain FBS (fetal bovine serum). This data shows that EGF is critical for primary intestinal cell barrier function in serum free media and also shows that hydrocortisone can compensate for a lack of FBS and help maintain primary intestinal cell barrier function. This is exemplified in that TEER had collapsed by day 8 in culture for media 2 (intestinal DM SF) but not for media 4 (intestinal DM SF and 1 uM hydrocortisone) which had stable TEER after 8 days in culture.

### Example 4-Selection of Media for the Co-Culture of Primary Liver Cells and Primary Intestinal Cells

In order to establish how primary liver cells and primary intestinal cells could be co-cultured without compromising liver cell metabolic functionality or intestinal barrier function, co-culture was performed in a dual organ plate as described herein with 5 different media:
Media 1-(Liver media): WEM+4% v/v cocktail B+500 nM hydrocortisone (HC)
Media 2—Advanced DMEM+4% v/v cocktail B+500 nM hydrocortisone (HC)+2.5 ng/mL EGF+500 nM A83-01+1.25 uM N-acetylcysteine (NAC)
Media 3—Advanced DMEM+4% v/v cocktail B+500 nM hydrocortisone (HC)+5 ng/ml EGF+500 nM A83-01+1.25 uM N-acetylcysteine (NAC)
Media 4—Advanced DMEM+4% v/v cocktail B+500 nM hydrocortisone (HC)+10 ng/ml EGF+500 nM A83-01+1.25 uM N-acetylcysteine (NAC)
Media 5—
   System volume (liver and basolateral compartments)—Advanced DMEM+4% v/v cocktail B+500 nM hydrocortisone (HC)+500 nM A83-01+1.25 uM N-acetylcysteine (NAC)
   Apical compartment-Advanced DMEM+4% v/v cocktail B+500 nM hydrocortisone (HC)+50 ng/ml EGF+500 nM A83-01+1.25 uM N-acetylcysteine (NAC)

Note that for media 2-4 the same media was used in the system volume (the liver and basolateral compartments) and the apical compartment. However, for media 5, two different types of media were used in the system volume and the apical compartment; both the apical compartment media and system volume media are made from the same base media, however, only the media of the apical compartment comprised EGF.

FIG. 4 shows that only the liver media control (media 1) and media 5 were suitable for maintaining primary liver cell metabolic functionality. This is shown by CYP3A4 metabolic rate, which remained above the 1 pmol/min/million cell threshold for both of these medias. Meanwhile, the metabolic rate of CYP3A4 fell below the threshold by day 6 in culture for media 2, 3 and 4 which all comprise EGF. This data shows that EGF compromises the metabolic functionality of primary liver cells.

FIG. 5 shows that intestinal barrier strength was maintained by media 2, 3, 4, 5 and intestinal differentiation media when primary intestinal cells were co-cultured with primary liver cells. Taken with the data shown in FIG. 4 it was clear that of the media tested only media 5 was suitable for the co-culture of primary liver cells and primary intestinal cells, with both primary liver cell metabolic functionality and primary intestinal cell barrier functionality being maintained. Ultimately, this shows that the presence of EGF at the apical surface of primary intestinal cells is critical for barrier function, while the absence of EGF is required for the maintenance of primary liver cell metabolic functionality.

The invention will now be further described with reference to the following clauses.
Clause 1. A method of co-culturing primary liver cells and primary intestinal cells, the method comprising;
   a. providing a system comprising at least one three compartment set, comprising a liver compartment, an apical compartment and a basolateral compartment, wherein the system comprises a first fluid circulation path whereby the basolateral compartment is fluidically connected to the liver compartment, the first fluid circulation path being selectively interruptible so as to fluidically isolate the liver compartment from the basolateral compartment;
   b. seeding primary liver cells in the liver compartment;
   c. adding primary intestinal cells to the apical compartment wherein said primary intestinal cells form a barrier between the apical and basolateral compartments;
   d. circulating media through the first fluid circulation paths that does not comprise EGF;
   e. adding media to the apical compartment that comprises EGF.
Clause 2. The method of clause 1 wherein the system further comprises a second fluid circulation path, wherein fluid in the liver compartment is recirculated.
Clause 3. The method of clause 1 or 2 wherein the system further comprises a third fluid circulation path, wherein fluid in the basolateral compartment is recirculated.
Clause 4. The method of any preceding clause wherein said primary liver cells are seeded on a 3D scaffold and/or said primary intestinal cells are seeded on a biomimetic scaffold.
Clause 5. The method of any of clauses 1 to 4 wherein said primary liver cells are seeded on a porous collagen coated scaffold.
Clause 6. The method of any preceding clause wherein said primary intestinal cells are seeded on a biomimetic scaffold.
Clause 7. The method of any preceding clause further comprising adding at least one treatment to at least one compartment.
Clause 8. The method of clause 7 comprising adding at least one treatment to the apical compartment and adding at least one treatment to the liver compartment.
Clause 9. The method of clause 7 or 8 wherein the treatment is selected from at least one of a drug, nutrient, vitamin, metabolite, cytokine, chemokine, carbohydrate, lipid, nucleic acid, hormone or peptide.
Clause 10. The method of any of clause 7 to 9 further comprising taking samples of media from at least one compartment periodically and measuring the concentration of said at least one treatment in said samples.
Clause 11. The method of clause 10 further comprising calculating oral bioavailability of at least one treatment using the formula:

$$Oral Bioavailability = \frac{area under the curve ( oral ) * dose ( IV )}{area under the curve ( IV ) * dose ( oral )}$$

Clause 12. The method of any preceding clause wherein the primary liver cells and primary intestinal cells are human.
Clause 13. A method of determining bioavailability, the method comprising;
   a. providing at least one system with at least two three compartment sets comprising a liver compartment, an apical compartment and a basolateral compartment, wherein the system comprises a first fluid circulation path whereby the basolateral compartment is fluidically connected to the liver compartment, a second fluid circulation path whereby fluid in the liver compartment is recirculated, a third fluid circulation path whereby fluid in the basolateral compartment may be recirculated;
   b. in the first three compartment set, seeding primary liver cells in the liver compartment and adding primary intestinal cells to the apical compartment wherein said primary intestinal cells form a barrier between the apical and basolateral compartments;
   c. in the second three compartment set, seeding primary liver cells in the liver compartment and not adding primary intestinal cells to the apical compartment;
   d. circulating medium that does not comprise EGF through the first, second and third fluid circulation paths of the first and second three compartment sets;
   e. adding medium that does comprise EGF to the apical compartment of the first three compartment set;
   f. adding at least one treatment to the apical compartment of the first three compartment set and separately adding at least one treatment to the liver compartment of the second three compartment set;
   g. taking samples periodically from the apical compartment and the liver compartment of the first and second three compartment sets;
   h. measuring the concentration of at least one treatment in said samples;
   i. calculating bioavailability using the formula;

$$Oral Bioavailability = \frac{area under the curve ( oral ) * dose ( IV )}{area under the curve ( IV ) * dose ( oral )}$$

Clause 14. A system for the co-culture of primary liver cells and primary intestinal cells, wherein said system comprises at least one three compartment set comprising a liver compartment comprising primary liver cells, an apical compartment comprising primary intestinal cells and a basolateral compartment, wherein said primary intestinal cells form a barrier between the apical and basolateral compartments and the system comprises a first fluid circulation path whereby the basolateral compartment is fluidically connected to the liver compartment, the first fluid circulation path being selectively interruptible so as to fluidically isolate the liver compartment from the basolateral compartment; the three compartment set may further comprise a second fluid circulation path whereby fluid in the liver compartment is recirculated, and/or a third fluid circulation path whereby fluid in the basolateral compartment may be recirculated, and only the media in the apical compartment comprises EGF.
Clause 15. A system according to clause 14 wherein media does not pass from the apical compartment to the liver compartment.
Clause 16. A system according to clause 14 or 15 wherein a porous membrane separates the apical compartment and the basolateral compartment.
Clause 17. A system according to any of clauses 14 to 16 wherein at least one compartment comprises a 3D scaffold.
Clause 18. A system according to clause 17 wherein the liver compartment comprises a 3D scaffold and/or and the apical compartment comprises a biomimetic scaffold.
Clause 19. A system according to any of clauses 17 or 18 wherein the liver compartment comprises a porous collagen coated scaffold.
Clause 20. A system according to any of clauses 14 to 19 wherein the apical compartment comprises primary jejunum stem/progenitor cells.
Clause 21. A system according to any of clauses 14 to 20 wherein the liver compartment comprises primary hepatocytes.
Clause 22. A system according to any of clauses 14 to 21 wherein the primary liver cells and primary intestinal cells are human.

1. A method of co-culturing primary liver cells and primary intestinal cells, the method comprising;
a. providing a system comprising at least one three compartment set, comprising a liver compartment, an apical compartment and a basolateral compartment, wherein the system comprises a first fluid circulation path whereby the basolateral compartment is fluidically connected to the liver compartment, the first fluid circulation path being selectively interruptible so as to fluidically isolate the liver compartment from the basolateral compartment;
b. seeding primary liver cells in the liver compartment;
c. adding primary intestinal cells to the apical compartment wherein said primary intestinal cells form a barrier between the apical and basolateral compartments;
d. circulating media through the first fluid circulation paths that does not comprise EGF;
e. adding media to the apical compartment that comprises EGF.

2. The method of claim 1 wherein the system further comprises a second fluid circulation path, wherein fluid in the liver compartment is recirculated.

3. The method of claim 1 wherein the system further comprises a third fluid circulation path, wherein fluid in the basolateral compartment is recirculated.

4. The method of claim 1 wherein said primary liver cells are seeded on a 3D scaffold and/or said primary intestinal cells are seeded on a biomimetic scaffold.

5. The method of claim 1 wherein said primary liver cells are seeded on a porous collagen coated scaffold.

6. The method of claim 1 wherein said primary intestinal cells are seeded on a biomimetic scaffold.

7. The method of claim 1 further comprising adding at least one treatment to at least one compartment.

8. The method of claim 7 comprising adding at least one treatment to the apical compartment and adding at least one treatment to the liver compartment.

9. The method of claim 7 wherein the treatment is selected from at least one of a drug, nutrient, vitamin, metabolite, cytokine, chemokine, carbohydrate, lipid, nucleic acid, hormone or peptide.

10. The method of claim 7 further comprising taking samples of media from at least one compartment periodically and measuring the concentration of said at least one treatment in said samples.

11. The method of claim 10 further comprising calculating oral bioavailability of at least one treatment using the formula:
$$Oral Bioavailability = \frac{area under the curve ( oral ) * dose ( IV )}{area under the curve ( IV ) * dose ( oral )}$$

12. The method of claim 1, wherein the primary liver cells and primary intestinal cells are human.

13. A method of determining bioavailability, the method comprising;
a. providing at least one system with at least two three compartment sets comprising a liver compartment, an apical compartment and a basolateral compartment, wherein the system comprises a first fluid circulation path whereby the basolateral compartment is fluidically connected to the liver compartment, a second fluid circulation path whereby fluid in the liver compartment is recirculated, a third fluid circulation path whereby fluid in the basolateral compartment may be recirculated;
b. in the first three compartment set, seeding primary liver cells in the liver compartment and adding primary intestinal cells to the apical compartment wherein said primary intestinal cells form a barrier between the apical and basolateral compartments;
c. in the second three compartment set, seeding primary liver cells in the liver compartment and not adding primary intestinal cells to the apical compartment;
d. circulating medium that does not comprise EGF through the first, second and third fluid circulation paths of the first and second three compartment sets;
e. adding medium that does comprise EGF to the apical compartment of the first three compartment set;
f adding at least one treatment to the apical compartment of the first three compartment set and separately adding at least one treatment to the liver compartment of the second three compartment set;
g. taking samples periodically from the apical compartment and the liver compartment of the first and second three compartment sets;
h. measuring the concentration of at least one treatment in said samples;
i. calculating bioavailability using the formula;
$$Oral Bioavailability = \frac{area under the curve ( oral ) * dose ( IV )}{area under the curve ( IV ) * dose ( oral )}$$

14. A system for the co-culture of primary liver cells and primary intestinal cells, wherein said system comprises at least one three compartment set comprising a liver compartment comprising primary liver cells, an apical compartment comprising primary intestinal cells and a basolateral compartment, wherein said primary intestinal cells form a barrier between the apical and basolateral compartments and the system comprises a first fluid circulation path whereby the basolateral compartment is fluidically connected to the liver compartment, the first fluid circulation path being selectively interruptible so as to fluidically isolate the liver compartment from the basolateral compartment; the three compartment set may further comprise a second fluid circulation path whereby fluid in the liver compartment is recirculated, and/or a third fluid circulation path whereby fluid in the basolateral compartment may be recirculated, and only the media in the apical compartment comprises EGF.

15. A system according to claim 14 wherein media does not pass from the apical compartment to the liver compartment.

16. A system according to claim 14 wherein a porous membrane separates the apical compartment and the basolateral compartment.

17. A system according to claim 14 wherein at least one compartment comprises a 3D scaffold.

18. A system according to claim 17 wherein the liver compartment comprises a 3D scaffold and/or and the apical compartment comprises a biomimetic scaffold.

19. A system according to claim 17 wherein the liver compartment comprises a porous collagen coated scaffold.

20. A system according to claim 14 wherein the apical compartment comprises primary jejunum stem/progenitor cells.

21. A system according to claim 14 wherein the liver compartment comprises primary hepatocytes.

22. A system according to claim 14 wherein the primary liver cells and primary intestinal cells are human.
